(19) United States
(12) Patent Application Publication
(10) Pub. No.: US 2026/0022110 A1
(43) Pub. Date: Jan. 22, 2026

(54) DIARYLACETYLENE COMPOUND, PREPARATION METHOD THEREOF, AND USES THEREOF

(71) Applicant: Henan Radiomedical Science and Technology Co., Ltd., Kaifeng (CN)

(72) Inventors: Marvin Xuejun XU, Kaifeng (CN); Yupo YANG, Kaifeng (CN); Zhengyan YANG, Kaifeng (CN); Chaoqun DUAN, Kaifeng (CN); Hongyun XU, Kaifeng (CN); Mengfu PEI, Kaifeng (CN); Yaqing LIU, Kaifeng (CN); Cen LI, Kaifeng (CN); Shanshun TANG, Kaifeng (CN)

(21) Appl. No.: 19/342,467

(22) Filed: Sep. 26, 2025

Related U.S. Application Data
(63) Continuation-in-part of application No. PCT/CN2024/082905, filed on Mar. 21, 2024.

(30) Foreign Application Priority Data
Apr. 19, 2023 (CN) 202310421271.3
Jun. 9, 2023 (CN) 202310682118.6

(51) Int. Cl. C07D 403/12; A61K 31/496; A61K 31/497; A61K 31/506; A61P 35/00; A61P 35/02; C07D 401/12; C07D 401/14; C07D 403/14; C07D 405/14

(52) U.S. Cl. CPC C07D 403/12; A61K 31/496; A61K 31/497; A61K 31/506; A61P 35/00; A61P 35/02; C07D 401/12; C07D 401/14; C07D 403/14; C07D 405/14

(57) ABSTRACT

The disclosure provides diarylacetylene compounds, their preparation methods, and applications. These compounds contain a biologically active isoindolinyl-piperazinyl urea core moiety. Further chemical modification of this core generates numerous compounds with higher biological activity, or their pharmaceutically acceptable salts, hydrates, solvates, metabolites, prodrugs, or pharmaceutical compositions. This expands the broad application potential of such compounds in biomedicine and the prospects for pharmaceutical formulation development. This class of compounds can target the STAT3 protein. At low doses (nanomolar concentrations), they significantly inhibit the proliferation of various tumor cells, including lung cancer, breast cancer, liver cancer, pancreatic cancer, and gastric cancer cells. They also effectively suppress the growth of transplanted tumors in mice. These results indicate that this class of compounds holds promise for development into drugs for the prevention and/or treatment of tumors and other diseases associated with abnormal STAT3 signaling.

### CROSS-REFERENCE TO RELATED APPLICATIONS

This application is a continuation-in-part of International Patent Application No. PCT/CN2024/082905 with an international filing date of Mar. 21, 2024, designating the United States, now pending, further claims foreign priority benefits to Chinese Patent Application No. 202310421271.3 filed Apr. 19, 2023, and to Chinese Patent Application No. 202310682118.6 filed Jun. 9, 2023. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, MA 02142.

### BACKGROUND

The disclosure relates to the field of tumor-targeted therapy, and specifically to a diarylacetylene compound, preparation method thereof, and uses thereof.

According to data released by the International Agency for Research on Cancer (IARC) under the World Health Organization (WHO), there were 19.3 million new cancer cases and 9.96 million cancer deaths globally in 2020. It is projected that by 2030, global annual new cancer cases will increase by 69% to 21 million, while cancer deaths will rise by 72%, increasing from 7.6 million in 2008 to 13 million. Worldwide, the prevention and treatment of cancer remain a formidable challenge.

Conventional antitumor drugs such as paclitaxel, doxorubicin, and vincristine offer broad-spectrum anticancer activity and proven efficacy but are associated with significant toxic side effects. Although diverse targeted cancer therapies exist clinically, they often suffer from low response rates or drug resistance, severely limiting treatment options for patients. Thus, the active exploration and development of novel anticancer agents are of critical clinical importance.

Signal Transducer and Activator of Transcription 3 (STAT3) is a core regulator of cellular signal transduction. In normal cells, STAT3 activation is typically transient; however, in approximately 70% of human malignancies, STAT3 is constitutively activated in response to cytokines, growth factors, and oncogenic signals. Activated STAT3 forms dimers via its SH2 domain, translocates to the nucleus, binds specific DNA sequences, and regulates the transcription of the entire repertoire of target genes, driving multiple procarcinogenic events. Such as:

Upregulating cyclin D1 or c-MYC proto-oncogene expression to promote tumor cell proliferation; Upregulating anti-apoptotic genes (BCL-2, BCL-XL, Survivin) to enhance tumor cell survival; Upregulating Vascular Endothelial Growth Factor (VEGF) to stimulate tumor angiogenesis; Upregulating IL-6, IL-10, Matrix Metalloproteinases (MMPs), and TGF-β to regulate Epithelial-Mesenchymal Transition (EMT) and Cancer Stem Cell (CSC)-like transformation.

Extensive studies demonstrate that STAT3 activation is significantly correlated with poor prognosis in lung, breast, gastric, liver, pancreatic, prostate cancers, leukemia, and others. Genetic knockdown or inhibition of STAT3 expression markedly suppresses tumor proliferation, survival, angiogenesis, drug resistance, immune evasion, metastasis, and recurrence. Therefore, STAT3-targeted therapies offer broad anticancer spectra and high efficacy.

### SUMMARY

This disclosure provides a novel class of diarylacetylene compounds with unique structural scaffolds. Biological analyses reveal that these compounds potently inhibit STAT3 activation and, at extremely low doses (nanomolar range), significantly suppress the proliferation of diverse tumor cells, including: Lung, breast, liver, pancreatic, gastric, thyroid cancers; glioma; head and neck, esophageal, cholangiocarcinoma, colorectal, testicular, thymoma, renal, prostate, bladder, uterine, ovarian cancers; retinoblastoma; mesothelioma; osteosarcoma; lymphoma; multiple myeloma; leukemia; chronic myelodysplastic syndrome; and melanoma. In breast cancer xenograft models, their efficacy is comparable to albumin-bound paclitaxel at equivalent doses, with no significant toxic side effects observed. Thus, these compounds hold promise for development as drugs to prevent and/or treat tumors and other diseases linked to abnormal STAT3 signaling.

The first objective of the disclosure is to provide a diarylacetylene compound having the following general formula (I):

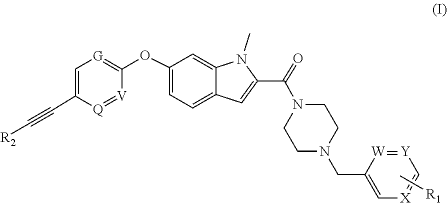

wherein R₁ is selected from the group consisting of H, F, Cl, Br, I, —CN, —CH₃, —CF₃, —OCH₃, —OCF₃,

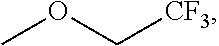

### and —SO₂NH;

R₂ is selected from the group consisting of

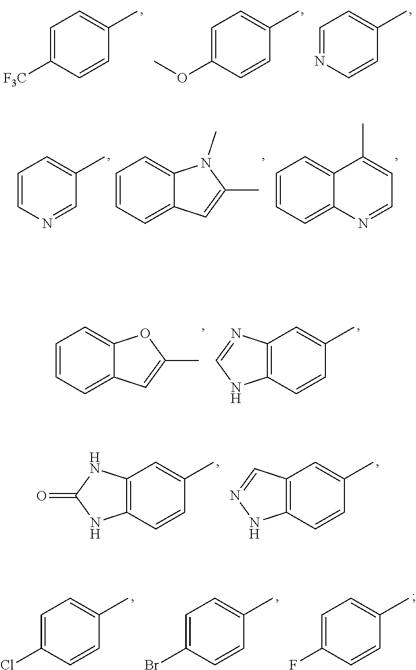

W, X, Y, G, Q, and V are each independently selected from the group consisting of C and N

In a class of this embodiment, the diarylacetylene compound is selected from the group consisting of the following formulas:

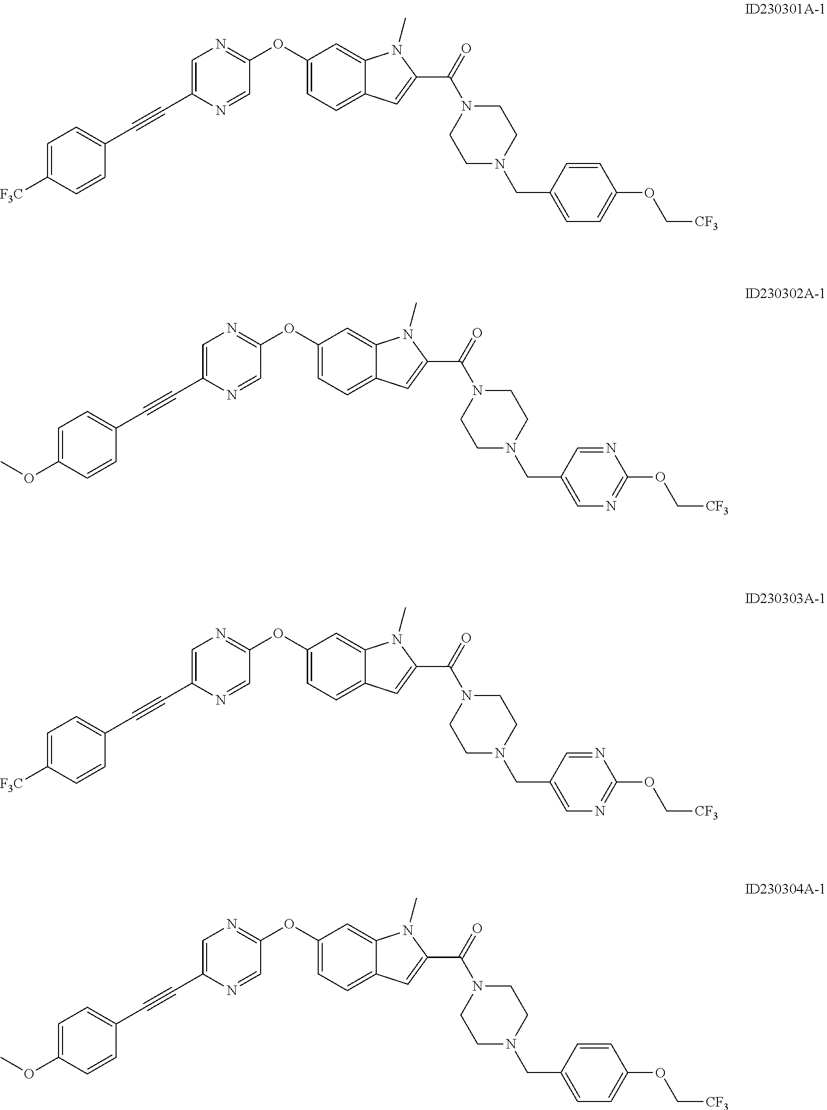 [SMILES: Cn1c(C(=O)N2CCN(Cc3ccc(OCC(F)(F)F)cc3)CC2)cc2ccc(Oc3cnc(C#Cc4ccc(C(F)(F)F)cc4)cn3)cc21 ; COc1ccc(C#Cc2cnc(Oc3ccc4cc(C(=O)N5CCN(Cc6cnc(OCC(F)(F)F)nc6)CC5)n(C)c4c3)cn2)cc1 ; Cn1c(C(=O)N2CCN(Cc3cnc(OCC(F)(F)F)nc3)CC2)cc2ccc(Oc3cnc(C#Cc4ccc(C(F)(F)F)cc4)cn3)cc21 ; COc1ccc(C#Cc2cnc(Oc3ccc4cc(C(=O)N5CCN(Cc6ccc(OCC(F)(F)F)cc6)CC5)n(C)c4c3)cn2)cc1]

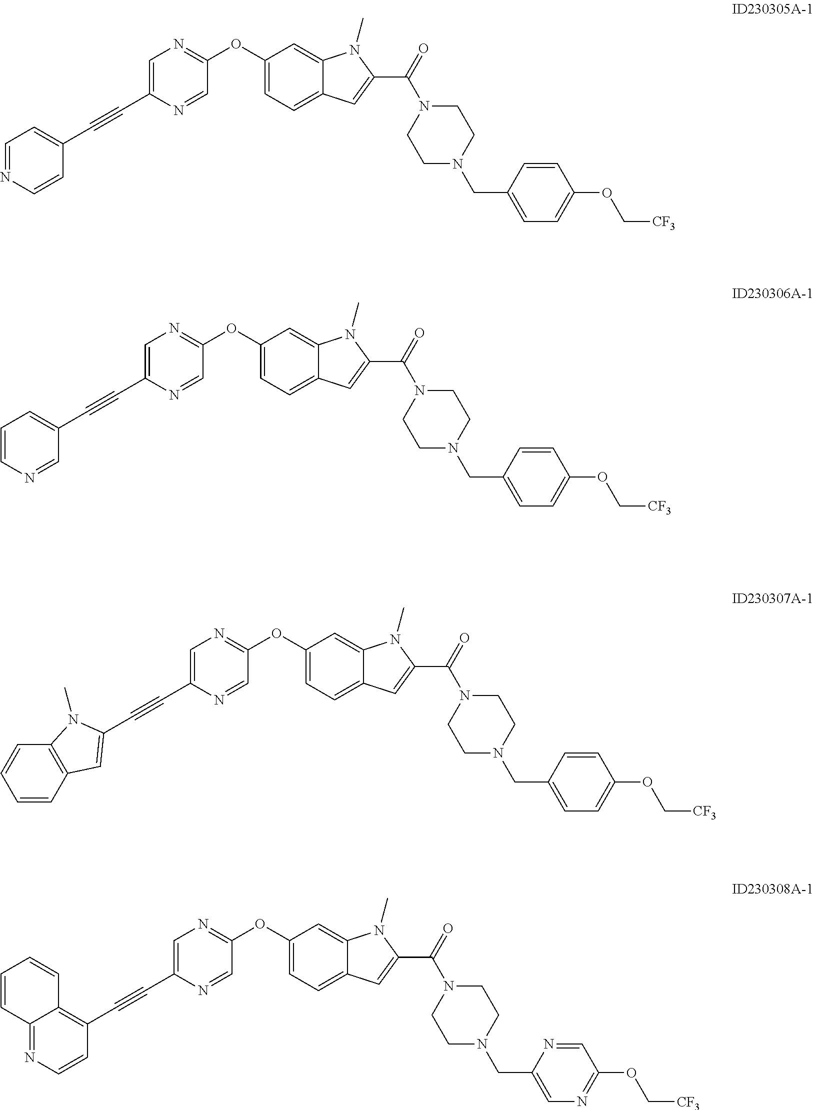 [SMILES: Cn1c(C(=O)N2CCN(Cc3ccc(OCC(F)(F)F)cc3)CC2)cc2ccc(Oc3cnc(C#Cc4ccncc4)cn3)cc21 ; Cn1c(C(=O)N2CCN(Cc3ccc(OCC(F)(F)F)cc3)CC2)cc2ccc(Oc3cnc(C#Cc4cccnc4)cn3)cc21 ; Cn1c(C#Cc2cnc(Oc3ccc4cc(C(=O)N5CCN(Cc6ccc(OCC(F)(F)F)cc6)CC5)n(C)c4c3)cn2)cc2ccccc21 ; Cn1c(C(=O)N2CCN(Cc3cnc(OCC(F)(F)F)cn3)CC2)cc2ccc(Oc3cnc(C#Cc4ccnc5ccccc45)cn3)cc21]

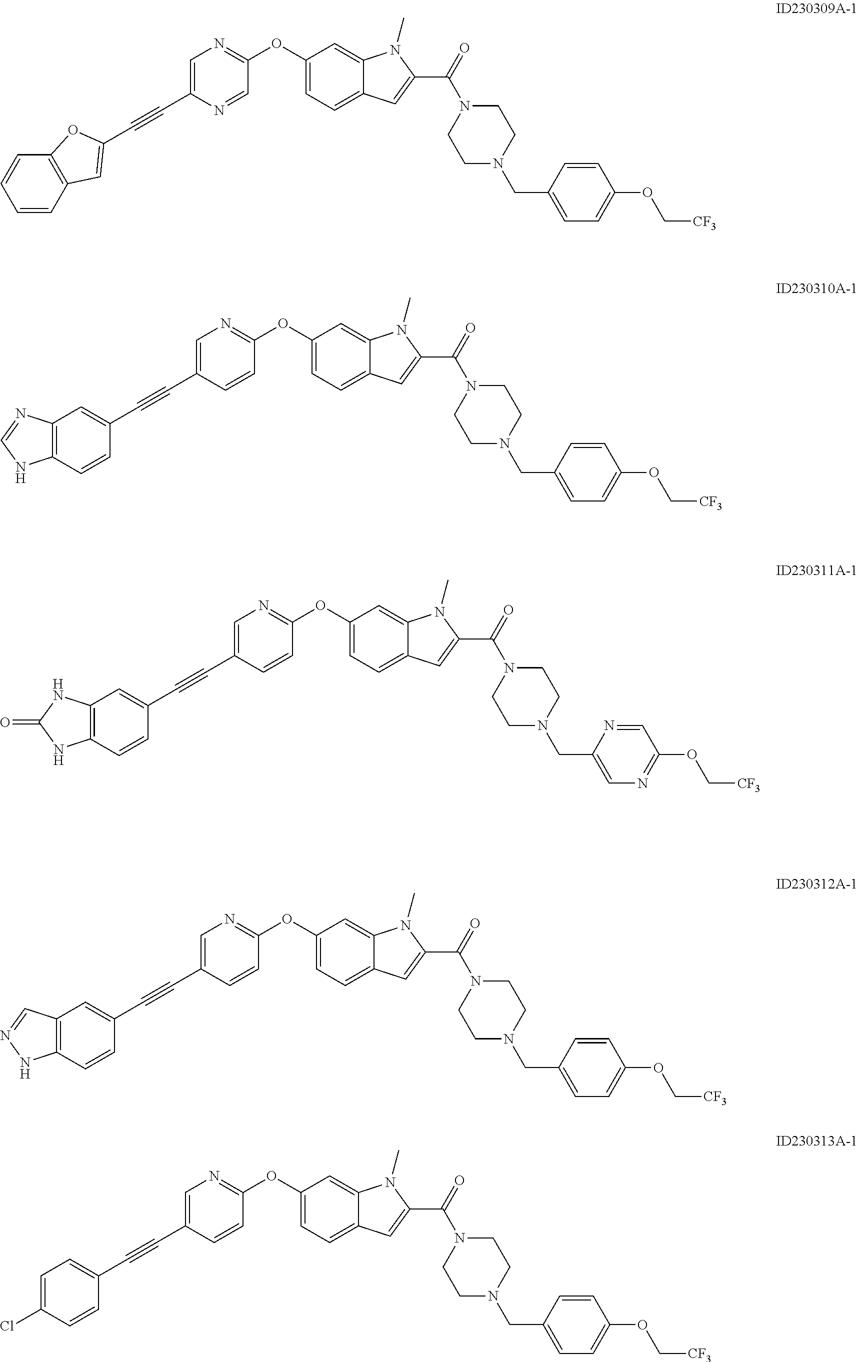 [SMILES: Cn1c(C(=O)N2CCN(Cc3ccc(OCC(F)(F)F)cc3)CC2)cc2ccc(Oc3cnc(C#Cc4cc5ccccc5o4)cn3)cc21 ; Cn1c(C(=O)N2CCN(Cc3ccc(OCC(F)(F)F)cc3)CC2)cc2ccc(Oc3ccc(C#Cc4ccc5[nH]cnc5c4)cn3)cc21 ; Cn1c(C(=O)N2CCN(Cc3cnc(OCC(F)(F)F)cn3)CC2)cc2ccc(Oc3ccc(C#Cc4ccc5[nH]c(=O)[nH]c5c4)cn3)cc21 ; Cn1c(C(=O)N2CCN(Cc3ccc(OCC(F)(F)F)cc3)CC2)cc2ccc(Oc3ccc(C#Cc4ccc5[nH]ncc5c4)cn3)cc21 ; Cn1c(C(=O)N2CCN(Cc3ccc(OCC(F)(F)F)cc3)CC2)cc2ccc(Oc3ccc(C#Cc4ccc(Cl)cc4)cn3)cc21]

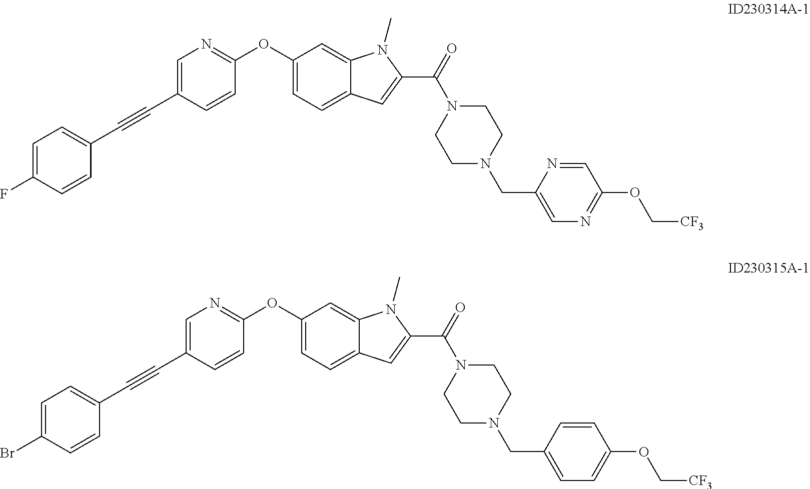 [SMILES: Cn1c(C(=O)N2CCN(Cc3cnc(OCC(F)(F)F)cn3)CC2)cc2ccc(Oc3ccc(C#Cc4ccc(F)cc4)cn3)cc21 ; Cn1c(C(=O)N2CCN(Cc3ccc(OCC(F)(F)F)cc3)CC2)cc2ccc(Oc3ccc(C#Cc4ccc(Br)cc4)cn3)cc21]

In a class of this embodiment, a pharmaceutically acceptable salt of a diarylacetylene compound is formed with at least one acid selected from the group consisting of acetic acid, dihydrofolic acid, benzoic acid, citric acid, sorbic acid, propionic acid, oxalic acid, fumaric acid, maleic acid, hydrochloric acid, malic acid, phosphoric acid, sulfurous acid, sulfuric acid, vanillic acid, tartaric acid, ascorbic acid, boric acid, lactic acid, and ethylenediaminetetraacetic acid.

The second objective of the disclosure is to provide a method for preparing the diarylacetylene compound with a synthetic route as follows:

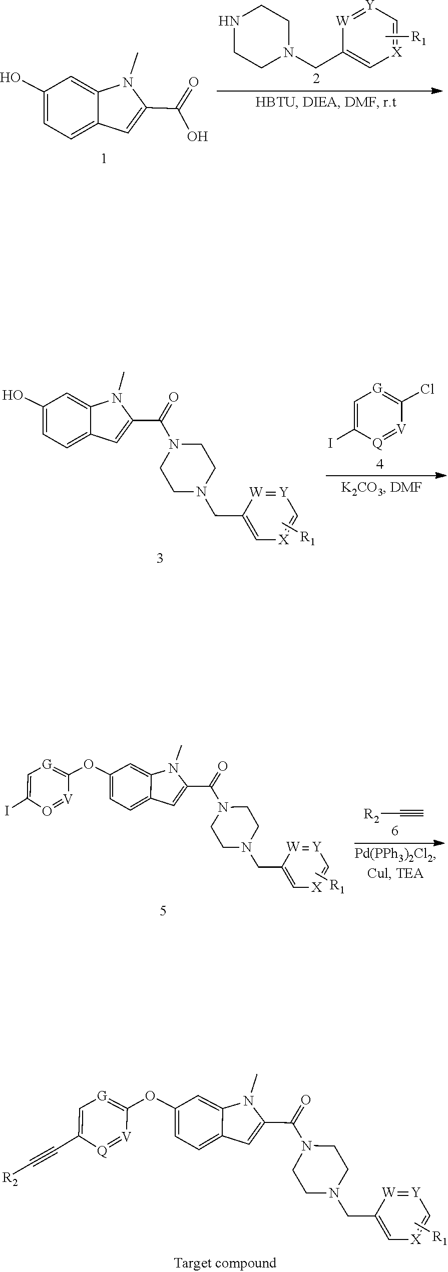

the method comprises:
(a) dissolving a first mixture of Compound 1, Compound 2, HBTU and DIEA in DMF, stirring the first mixture at a temperature of 20° C. to 30° C., diluting the first mixture with EA and washing with brine, collecting a first organic layer and concentrating under reduced pressure to yield a first crude product, and triturating the first crude product with EA to yield Compound 3;
(b) dissolving a second mixture of Compound 3, Compound 4 and K₂CO₃ in DMF, stirring the second mixture at a temperature of 75° C. to 85° C., diluting the second mixture with EA and washing with brine, collecting a second organic layer and concentrating under reduced pressure to yield a second crude product, and triturating the second crude product with EA to yield Compound 5; and
(c) stirring a third mixture of Compound 5, Compound 6, Pd(PPh₃)₂Cl₂, CuI, and TEA under a nitrogen atmosphere at a temperature of 75° C. to 85° C., diluting the third mixture with EA and washing with brine, collecting a third organic layer and concentrating under reduced pressure to yield a third crude product, triturating the third crude product with EA to yield a target compound.

In a class of this embodiment, in (a), the molar ratio of Compound 1 to Compound 2 to HBTU to DIEA is 1:1:1.2:3; in (b), the molar ratio of Compound 3 to Compound 4 to K₂CO₃ is 1:1:1.2; in (c), the molar ratio of Compound 5 to Compound 6 to Pd(PPh₃)₂Cl₂ to CuI to TEA is 1:5:0.1:0.2:5.

The third objective of the disclosure is to provide a method for treating a disease associated with STAT3 signaling, the method comprising administering a subject in need thereof the compound or a pharmaceutically acceptable salt thereof.

In a class of this embodiment, the disease is cancer.

In a class of this embodiment, the cancer is selected from the group consisting of lung cancer, breast cancer, liver cancer, pancreatic cancer, gastric cancer, thyroid cancer, glioma, head and neck cancer, esophageal cancer, cholangiocarcinoma, colorectal cancer, testicular cancer, thymoma, renal cancer, prostate cancer, bladder cancer, uterine cancer, ovarian cancer, retinoblastoma, mesothelioma, osteosarcoma, lymphoma, multiple myeloma, leukemia, chronic myelodysplastic syndrome, and melanoma.

In a class of this embodiment, the diarylacetylene compound includes, but is not limited to: ID230301A-1, ID230302A-1, ID230303A-1, ID230304A-1, ID230305A-1, ID230306A-1, ID230307A-1, ID230308A-1, ID230309A-1, ID230310A-1, ID230311A-1, ID230312A-1, ID230313A-1, ID230314A-1, and ID230315A-1. Antiproliferative activity of the diarylacetylene compound against multiple cancer cell lines was evaluated using the CCK-8 assay. Effects on in vivo tumor growth were assessed in breast cancer xenograft models in nude mice.

In a class of this embodiment, the diarylacetylene compound and a pharmaceutically acceptable salt thereof were evaluated for antiproliferative activity against a variety of tumor cell lines, including lung cancer, breast cancer, liver cancer, pancreatic cancer, gastric cancer, thyroid cancer, glioma, head and neck cancer, esophageal cancer, cholangiocarcinoma, colorectal cancer, testicular cancer, thymoma, renal cancer, prostate cancer, bladder cancer, uterine cancer, ovarian cancer, retinoblastoma, mesothelioma, osteosarcoma, lymphoma, multiple myeloma, leukemia, and chronic myelodysplastic syndrome.

Accordingly, the disclosure provides the diarylacetylene compound and a pharmaceutically acceptable salt thereof for use in methods of treating diseases associated with STAT3 signaling, including cancers as listed above.

### BRIEF DESCRIPTION OF THE DRAWINGS

FIG. 1A is a graph showing suppression of MDA-MB-468 xenograft growth in mice treated with ID230301A-1 or paclitaxel compared to vehicle-treated controls.

FIG. 1B is a graph showing quantitative end-point analysis of tumor volume demonstrating superior antitumor efficacy of ID230301A-1 versus paclitaxel.

FIG. 1C is a graph showing body weight changes of mice at study termination, indicating preservation of body mass by ID230301A-1 compared to weight loss caused by paclitaxel.

FIG. 2 is a Western blot showing the inhibition of STAT3 phosphorylation by ID230301A-1.

### DETAILED DESCRIPTION

To clarify the technical objectives, solutions, and beneficial effects of the disclosure, the technical schemes are further described below with reference to the drawings and specific examples.

### General Synthetic Guidelines:

Starting materials for preparing Formula (I) compounds:
Commercially available;
Prepared by literature-known methods; or
Synthesized via techniques accessible to skilled artisans.
Modifiable parameters:

Intermediates, reagents, reaction conditions may be adjusted per empirical knowledge.

### Standard Experimental Conventions:

(i) Temperature: Reactions conducted at room temperature (20-30° C.) unless specified;
(ii) Solvents:
Organic solvents dried using standard techniques;
Concentrated by rotary evaporation (bath temperature <50° C.);
Eluents/solvent mixtures reported as volume/volume ratios;
(iii) Reaction Monitoring: By thin-layer chromatography (TLC);
(iv) Product Validation: By satisfactory ¹H-NMR spectra.

### Example 1

### Synthesis of the Diarylacetylene Compound Followed the Route Below

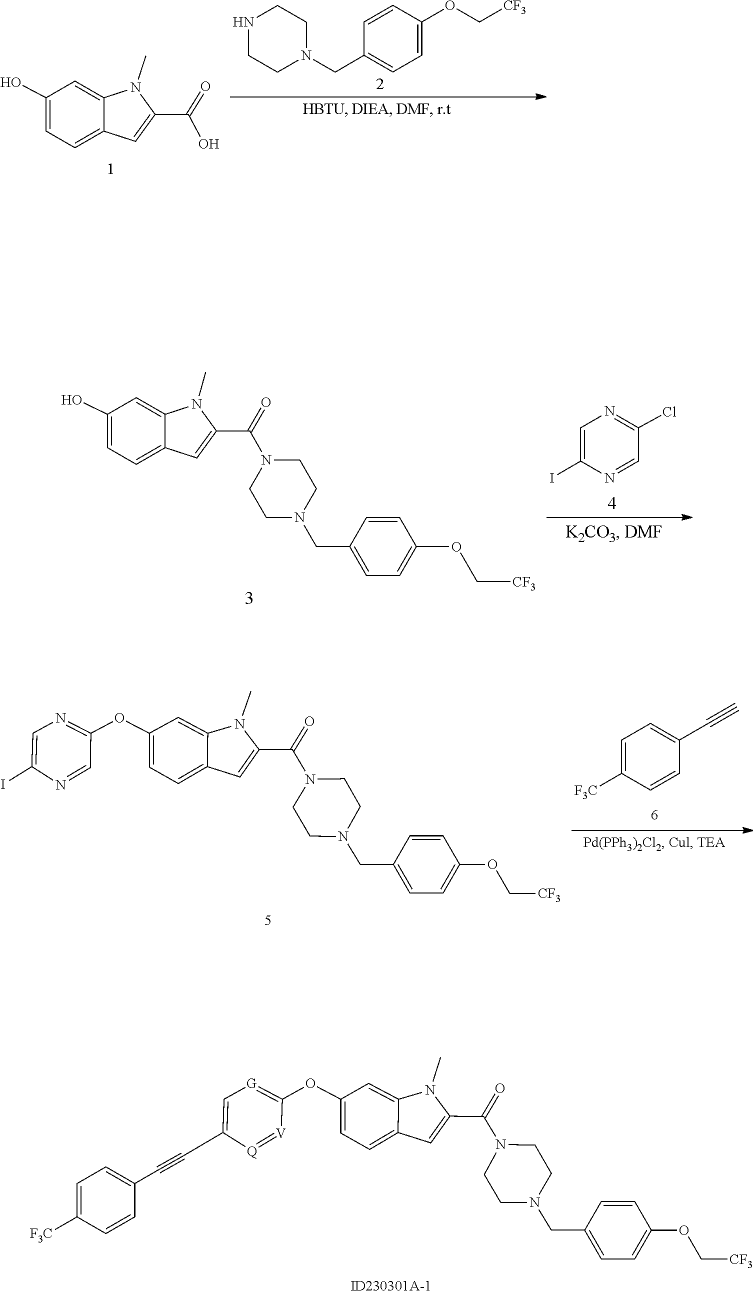

Specific synthesis method, taking Compound ID230301A-1 as an example, with the structural formula as follows.

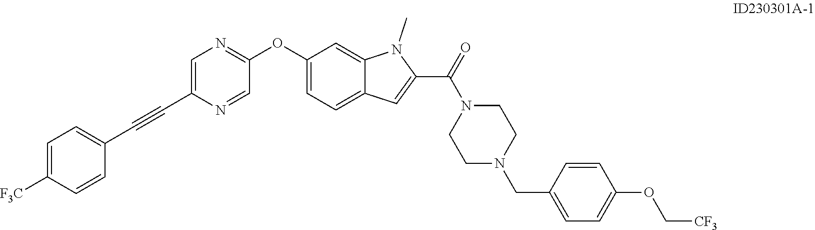 [SMILES: Cn1c(C(=O)N2CCN(Cc3ccc(OCC(F)(F)F)cc3)CC2)cc2ccc(Oc3cnc(C#Cc4ccc(C(F)(F)F)cc4)cn3)cc21]

The chemical name of Compound ID230301A-1 is: (1-methyl-6-((5-((4-(trifluoromethyl)phenyl)ethynyl)pyrazin-2-yl)oxy)-1H-indol-2-yl) (4-(4-(2,2,2-trifluoroethoxy)benzyl)piperazin-1-yl)methanone.

### Step 1. Preparation of (6-hydroxy-1-methyl-1H-indol-2-yl)(4-(4-(2,2,2-trifluoroethoxy)benzyl)piperazin-1-yl)methanone (Compound 3)

A mixture of Compound 1 (5.0 g, 26.15 mmol, 1.0 eq), Compound 2(7.17 g, 26.15 mmol, 1.0 eq), HBTU (14.90 g, 31.38 mmol, 1.2 eq) and DIEA (10.14 g, 78.46 mmol, 3.0 eq) in DMF (50 mL) was stirred at 25° C. for 3 h. TLC indicated that the reaction was completed. The reaction mixture was diluted with ethyl acetate (EA, 500 mL), washed with brine for 3 times (3×400 mL), dried over anhydrous sulfate sodium, and concentrated under reduce pressure. The crude product was triturated with EA (25 mL) to yield 8.5 g of Compound 3 as a yellow solid (yield: 72.6%).

1H NMR (DMSO-d⁶, 300 MHz) δ: 11.16 (s, 1H), 9.19 (s, 1H), 7.38 (d, J=8.6 Hz, 1H), 7.31 (d, J=8.7 Hz, 2H), 7.05 (d, J=8.7 Hz, 2H), 6.78 (d, J=2.0 Hz, 1H), 6.68 (d, J=1.4 Hz, 1H), 6.59 (dd, J=8.6, 2.1 Hz, 1H), 4.77 (q, J=8.9 Hz, 2H), 3.76 (s, 4H), 3.72 (s, 3H), 3.50 (s, 2H), 2.46-2.41 (m, 4H).

### Step 2. Preparation of (4-(2-(piperidin-1-yl)ethoxy)phenyl)methanol (Compound 5)

A mixture of Compound 3 (5.0 g, 11.17 mmol, 1.0 eq), Compound 4 (2.69 g, 11.17 mmol, 1.0 eq) and K₂CO₃ (1.85 g, 13.41 mmol, 1.2 eq) in DMF (50 mL) was stirred at 80° C. for 2 h. TLC indicated that the reaction was completed. The reaction mixture was diluted with EA (300 mL) and washed with brine for 3 times (3×200 mL), dried over anhydrous sulfate sodium, and concentrated under reduce pressure. The crude product was triturated with EA (18 mL) to yield 5.20 g compound 5 as a yellow solid (yield: 71.4%).

### Step 3. Preparation of (1-methyl-6-((5-((4-(trifluoromethyl)phenyl)ethynyl)pyrazin-2-yl)oxy)-1H-indol-2-yl) (4-(4-(2,2,2-trifluoroethoxy)benzyl)piperazin-1-yl)methanone (ID230301A-1)

A mixture of Compound 5(1.0 g, 1.54 mmol, 1.0 eq), Compound 6(1.31 g, 7.68 mmol, 5.0 eq), Pd(PPh₃)₂Cl₂(107 mg, 0.15 mmol, 0.10 eq), CuI(58.4 mg, 0.30 mmol, 0.2 eq) and TEA(776 mg, 7.68 mmol, 5.0 eq) in DMF (20 mL) was stirred at 80° C. for 48 h under N₂ atmosphere. TLC indicated that the reaction was completed. The reaction mixture was diluted with EA (300 mL) and washed with brine for 3 times (3×200 mL), dried over anhydrous sulfate sodium, and concentrated under reduce pressure. The crude product was purified by silica gel column chromatography (DCM/MeOH=70/1˜20/1) to yield 480 mg of ID230301A-1 as a yellow solid (yield: 45.2%).

1H NMR (CDCl₃, 400 MHz) δ: 9.01 (s, 1H), 8.60 (s, 1H), 8.16 (d, J=8.7 Hz, 2H), 7.69 (d, J=8.6 Hz, 1H), 7.32 (d, J=8.0 Hz, 2H), 7.24 (s, 1H), 7.20 (d, J=8.8 Hz, 2H), 6.99 (dd, J=8.5, 1.8 Hz, 1H), 6.94 (d, J=8.4 Hz, 2H), 6.64 (s, 1H), 4.37 (q, J=8.1 Hz, 2H), 3.87-3.77 (m, 7H), 3.56-3.54 (m, 2H), 2.53-2.50 (m, 4H).

### Example 2

### Antiproliferative Activity of ID230301A-1 to ID230315A-1 Against Tumor Cells

The antiproliferative activities of compounds ID230301A-1, ID230302A-1, ID230303A-1, ID230304A-1, ID230305A-1, ID230306A-1, ID230307A-1, ID230308A-1, ID230309A-1, ID230310A-1, ID230311A-1, ID230312A-1, ID230313A-1, ID230314A-1, and ID230315A-1 were evaluated in multiple tumor cell lines, including Lung carcinoma (H460), breast carcinoma (MDA-MB-468), hepatocellular carcinoma (HepG2), pancreatic carcinoma (BxPC-3), gastric carcinoma (MKN-45), thyroid carcinoma (TPC-1), glioma (U251), head and neck carcinoma (CNE-1), esophageal carcinoma (TE-11), cholangiocarcinoma (HuH28), colorectal (SW480), testicular carcinoma (NTERA-2 cl.D1), thymoma (Thy0517), renal carcinoma (GRC-1), prostate carcinoma (PC-3), bladder carcinoma (EJ-1), cervical carcinoma (HeLa), ovarian carcinoma (SKOV3), retinoblastoma (WERI-Rb-1), mesothelioma (MPP-89), osteosarcoma (U20S), lymphoma (Jurkat), multiple myeloma (MM1.S), leukemia (U937), chronic myelodysplastic syndrome (SKM-1), and melanoma (MEL202).

### Experimental Procedure

Cells in logarithmic growth phase were harvested, counted, and resuspended at 5×10⁴ cells/mL.

100 μL of cell suspension was seeded per well in 96-well plates.

Compounds were diluted in DMSO and added to wells to achieve final concentrations of 0.01, 0.03, 0.1, 0.3, 1, 3, 10, 30, and 100 mol/L.

Control wells received DMSO only.

After incubation for 48 h, 10 μL of CCK-8 reagent was added to each well.

Plates were incubated at 37° C. for 1 h.

Absorbance (OD₄₅₀ nm) was measured using a microplate reader.

Dose-response curves were plotted (compound concentration vs. OD₄₅₀).

Statistical Analysis: The half-maximal inhibitory concentration (IC₅₀) values were determined from the dose-response curves. Representative results are summarized in Tables 1-4.

TABLE 1
CCK-8 Assay Results for Inhibition of Lung, Breast, Liver, Pancreatic, Gastric,
and Thyroid Cancer Cell Proliferation by ID230301A-1-ID230315A-1

| | Tumor Cell | | | | | |
|---|---|---|---|---|---|---|
| | Lung | Breast | Liver | Pancreatic | Gastric | Thyroid |
| | cancer | cancer | cancer | cancer | cancer | cancer |
| Compound | H460 | MDA-MB-468 | HepG2 | BxPC-3 | MKN-45 | TPC-1 |
| ID230301A-1 | 0.0167 | 0.0044 | 0.0277 | 0.0174 | 0.0026 | 0.0065 |
| IC50(μM) | | | | | | |
| ID230302A-1 | 0.0762 | 0.0074 | 0.0262 | 0.0393 | 0.0042 | 0.0083 |
| IC50(μM) | | | | | | |
| ID230303A-1 | 0.0683 | 0.018 | 0.0526 | 0.0616 | 0.0379 | 0.0172 |
| IC50(μM) | | | | | | |
| ID230304A-1 | 0.0312 | 0.0914 | 0.0446 | 0.0671 | 0.0508 | 0.0491 |
| IC50(μM) | | | | | | |
| ID230305A-1 | 0.2506 | 0.0936 | 0.0071 | 0.0349 | 0.0123 | 0.1053 |
| IC50(μM) | | | | | | |
| ID230306A-1 | 0.0815 | 0.0221 | 0.059 | 0.0441 | 0.0052 | 0.0174 |
| IC50(μM) | | | | | | |
| ID230307A-1 | 0.0253 | 0.018 | 0.048 | 0.0546 | 0.0114 | 0.0069 |
| IC50(μM) | | | | | | |
| ID230308A-1 | 0.0618 | 0.0041 | 0.0213 | 0.0634 | 0.0601 | 0.0532 |
| IC50(μM) | | | | | | |
| ID230309A-1 | 0.0619 | 0.0511 | 0.0982 | 0.0142 | 0.0152 | 0.0417 |
| IC50(μM) | | | | | | |
| ID230310A-1 | 0.0432 | 0.0936 | 0.0909 | 0.0382 | 0.0662 | 0.0432 |
| IC50(μM) | | | | | | |
| ID230311A-1 | 0.1536 | 0.0124 | 0.0379 | 0.0547 | 0.0496 | 0.0387 |
| IC50(μM) | | | | | | |
| ID230312A-1 | 0.0462 | 0.0369 | 0.0015 | 0.0029 | 0.0745 | 0.0651 |
| IC50(μM) | | | | | | |
| ID230313A-1 | 0.0825 | 0.0045 | 0.0484 | 0.0326 | 0.0205 | 0.0112 |
| IC50(μM) | | | | | | |
| ID230314A-1 | 0.0568 | 0.0926 | 0.0379 | 0.0353 | 0.0527 | 0.0383 |
| IC50(μM) | | | | | | |
| ID230315A-1 | 0.0614 | 0.0177 | 0.0178 | 0.0949 | 0.0053 | 0.0243 |
| IC50(μM) | | | | | | |

TABLE 2
CCK-8 Assay Results for Inhibitory Effects of ID230301A-1-ID230315A-1 on Proliferation of Glioma,
Head and Neck, Esophageal, Cholangiocarcinoma, Colorectal, Testicular, and Thymoma Cells

| | Tumor Cell | | | | | | |
|---|---|---|---|---|---|---|---|
| | | Head and | | | | | |
| | | neck | Esophageal | Cholangio- | Colorectal | Testicular | |
| | Glioma | cancer | cancer | carcinoma | cancer | cancer | Thymoma |
| Compound | U251 | CNE-1 | TE-11 | HuH28 | SW480 | NTERA-2 cl.D1 | Thy0517 |
| ID230301A-1 | 0.0167 | 0.0044 | 0.0277 | 0.0174 | 0.0026 | 0.0091 | 0.0053 |
| IC50(μM) | | | | | | | |
| ID230302A-1 | 0.0762 | 0.0074 | 0.0262 | 0.0393 | 0.0042 | 0.0387 | 0.0192 |
| IC50(μM) | | | | | | | |
| ID230303A-1 | 0.0683 | 0.018 | 0.0526 | 0.0616 | 0.0379 | 0.0261 | 0.0547 |
| IC50(μM) | | | | | | | |
| ID230304A-1 | 0.0312 | 0.0914 | 0.0446 | 0.0671 | 0.0508 | 0.0413 | 0.0626 |
| IC50(μM) | | | | | | | |
| ID230305A-1 | 0.2506 | 0.0936 | 0.0071 | 0.0349 | 0.0123 | 0.0093 | 0.0158 |
| IC50(μM) | | | | | | | |
| ID230306A-1 | 0.0815 | 0.0221 | 0.059 | 0.0441 | 0.0052 | 0.0236 | 0.0087 |
| IC50(μM) | | | | | | | |
| ID230307A-1 | 0.0253 | 0.018 | 0.048 | 0.0546 | 0.0114 | 0.0034 | 0.0219 |
| IC50(μM) | | | | | | | |
| ID230308A-1 | 0.0618 | 0.0041 | 0.0213 | 0.0634 | 0.0601 | 0.0709 | 0.0053 |
| IC50(μM) | | | | | | | |
| ID230309A-1 | 0.0619 | 0.0511 | 0.0982 | 0.0142 | 0.0152 | 0.0317 | 0.0452 |
| IC50(μM) | | | | | | | |
| ID230310A-1 | 0.0432 | 0.0936 | 0.0909 | 0.0382 | 0.0662 | 0.0383 | 0.0751 |
| IC50(μM) | | | | | | | |
| ID230311A-1 | 0.1536 | 0.0124 | 0.0379 | 0.0547 | 0.0496 | 0.0372 | 0.0536 |
| IC50(μM) | | | | | | | |
| ID230312A-1 | 0.0462 | 0.0369 | 0.0015 | 0.0029 | 0.0745 | 0.0481 | 0.0719 |
| IC50(μM) | | | | | | | |
| ID230313A-1 | 0.0825 | 0.0045 | 0.0484 | 0.0326 | 0.0205 | 0.0153 | 0.0407 |
| IC50(μM) | | | | | | | |
| ID230314A-1 | 0.0568 | 0.0926 | 0.0379 | 0.0353 | 0.0527 | 0.0469 | 0.0287 |
| IC50(μM) | | | | | | | |
| ID230315A-1 | 0.0614 | 0.0177 | 0.0178 | 0.0949 | 0.0053 | 0.0493 | 0.0321 |
| IC50(μM) | | | | | | | |

TABLE 3
CCK-8 Assay Results for Inhibitory Effects of ID230301A-1-
ID230315A-1 on Proliferation of Renal, Prostate, Bladder,
Uterine, Ovarian, Retinoblastoma, and Mesothelioma Cells

| | Tumor Cell | | | | | | |
|---|---|---|---|---|---|---|---|
| | Renal cell | Prostate | Bladder | Uterine | Ovarian | Retino- | Meso- |
| | carcinoma | cancer | cancer | cancer | cancer | blastoma | thelioma |
| Compound | GRC-1 | PC-3 | EJ-1 | Hela | SKOV3 | WERI-Rb-1 | MPP-89 |
| ID230301A-1 | 0.0167 | 0.0044 | 0.0277 | 0.0174 | 0.0026 | 0.0019 | 0.0026 |
| IC50(μM) | | | | | | | |
| ID230302A-1 | 0.0762 | 0.0074 | 0.0262 | 0.0393 | 0.0042 | 0.0332 | 0.0146 |
| IC50(μM) | | | | | | | |
| ID230303A-1 | 0.0683 | 0.018 | 0.0526 | 0.0616 | 0.0379 | 0.0094 | 0.0218 |
| IC50(μM) | | | | | | | |
| ID230304A-1 | 0.0312 | 0.0914 | 0.0446 | 0.0671 | 0.0508 | 0.0478 | 0.0591 |
| IC50(μM) | | | | | | | |
| ID230305A-1 | 0.2506 | 0.0936 | 0.0071 | 0.0349 | 0.0123 | 0.0063 | 0.0237 |
| IC50(μM) | | | | | | | |
| ID230306A-1 | 0.0815 | 0.0221 | 0.0591 | 0.0441 | 0.0052 | 0.0071 | 0.0169 |
| IC50(μM) | | | | | | | |
| ID230307A-1 | 0.0253 | 0.0181 | 0.048 | 0.0546 | 0.0114 | 0.0602 | 0.0084 |
| IC50(μM) | | | | | | | |
| ID230308A-1 | 0.0618 | 0.0041 | 0.0213 | 0.0634 | 0.0601 | 0.0703 | 0.0312 |
| IC50(μM) | | | | | | | |
| ID230309A-1 | 0.0619 | 0.0511 | 0.0982 | 0.0142 | 0.0152 | 0.0854 | 0.0356 |
| IC50(μM) | | | | | | | |
| ID230310A-1 | 0.0432 | 0.0936 | 0.0909 | 0.0382 | 0.0662 | 0.0457 | 0.0712 |
| IC50(μM) | | | | | | | |
| ID230311A-1 | 0.1536 | 0.0124 | 0.0379 | 0.0547 | 0.0496 | 0.0383 | 0.0285 |
| IC50(μM) | | | | | | | |
| ID230312A-1 | 0.0462 | 0.0369 | 0.0015 | 0.0029 | 0.0745 | 0.0695 | 0.0403 |
| IC50(μM) | | | | | | | |
| ID230313A-1 | 0.0825 | 0.0045 | 0.0484 | 0.0326 | 0.0205 | 0.0311 | 0.0524 |
| IC50(μM) | | | | | | | |
| ID230314A-1 | 0.0568 | 0.0926 | 0.0379 | 0.0353 | 0.0527 | 0.0369 | 0.0175 |
| IC50(μM) | | | | | | | |
| ID230315A-1 | 0.0614 | 0.0177 | 0.0178 | 0.0949 | 0.0053 | 0.00302 | 0.0096 |
| IC50(μM) | | | | | | | |

TABLE 4
CCK-8 Assay Results for Inhibitory Effects of ID230301A-1-
ID230315A-1 on Osteosarcoma, Lymphoma, Multiple myeloma, Leukemia,
Chronic myelodysplastic syndrome, and Melanoma cells

| | Tumor Cell | | | | | |
|---|---|---|---|---|---|---|
| | | | | | Chronic | |
| | | | | | myelo- | |
| | Osteo- | | Multiple | | dysplastic | |
| | sarcoma | Lymphoma | myeloma | Leukemia | syndrome | Melanoma |
| Compound | U2OS | Jurkat | MM1.S | U937 | SKM-1 | MEL202 |
| ID230301A-1 | 0.0167 | 0.0044 | 0.0277 | 0.0174 | 0.0026 | 0.0136 |
| IC50(μM) | | | | | | |
| ID230302A-1 | 0.0762 | 0.0074 | 0.0262 | 0.0393 | 0.0042 | 0.0562 |
| IC50(μM) | | | | | | |
| ID230303A-1 | 0.0683 | 0.018 | 0.0526 | 0.0616 | 0.0379 | 0.0543 |
| IC50(μM) | | | | | | |
| ID230304A-1 | 0.0312 | 0.0914 | 0.0446 | 0.0671 | 0.0508 | 0.0418 |
| IC50(μM) | | | | | | |
| ID230305A-1 | 0.2506 | 0.0936 | 0.0071 | 0.0349 | 0.0123 | 0.1075 |
| IC50(μM) | | | | | | |
| ID230306A-1 | 0.0815 | 0.0221 | 0.059 | 0.0441 | 0.0052 | 0.0049 |
| IC50(μM) | | | | | | |
| ID230307A-1 | 0.0253 | 0.018 | 0.048 | 0.0546 | 0.0114 | 0.0231 |
| IC50(μM) | | | | | | |
| ID230308A-1 | 0.0618 | 0.0041 | 0.0213 | 0.0634 | 0.0601 | 0.0559 |
| IC50(μM) | | | | | | |
| ID230309A-1 | 0.0619 | 0.0511 | 0.0982 | 0.0142 | 0.0152 | 0.0144 |
| IC50(μM) | | | | | | |
| ID230310A-1 | 0.0432 | 0.0936 | 0.0909 | 0.0382 | 0.0662 | 0.0387 |
| IC50(μM) | | | | | | |
| ID230311A-1 | 0.1536 | 0.0124 | 0.0379 | 0.0547 | 0.0496 | 0.1352 |
| IC50(μM) | | | | | | |
| ID230312A-1 | 0.0462 | 0.0369 | 0.0015 | 0.0029 | 0.0745 | 0.0635 |
| IC50(μM) | | | | | | |
| ID230313A-1 | 0.0825 | 0.0045 | 0.0484 | 0.0326 | 0.0205 | 0.0422 |
| IC50(μM) | | | | | | |
| ID230314A-1 | 0.0568 | 0.0926 | 0.0379 | 0.0353 | 0.0527 | 0.0391 |
| IC50(μM) | | | | | | |
| ID230315A-1 | 0.0614 | 0.0177 | 0.0178 | 0.0949 | 0.0053 | 0.0276 |
| IC50(μM) | | | | | | |

The data in Tables 1, 2, 3, and 4 show that ID230301A-1, ID230302A-1, ID230303A-1, ID230304A-1, ID230305A-1, ID230306A-1, ID230307A-1, ID230308A-1, ID230309A-1, ID230310A-1, ID230311A-1, ID230312A-1, ID230313A-1, ID230314A-1, and ID230315A-1 have good proliferation inhibitory effects on lung cancer, breast cancer, liver cancer, pancreatic cancer, stomach cancer, thyroid cancer, glioma, cervical cancer, esophageal cancer, bile duct cancer, colon cancer, testicular cancer, thymoma, renal cancer, prostate cancer, bladder cancer, uterine cancer, ovarian cancer, retinoblastoma, mesothelioma, osteosarcoma, lymphoma, multiple osteomyeloma, leukemia, chronic bone marrow proliferation abnormality syndrome, and melanoma cell strains. The in vitro anti-tumor effects of such compounds have been further studied in the disclosure using ID230301A-1 as an example.

### Example 3

### Antitumor Efficacy of ID230301A-1 in Mice

Twenty female BALB/c-nude mice (6 weeks old, 18-22 g, SPF grade) were purchased from SPF (Beijing, China) Biotechnology Co., Ltd. and used to establish orthotopic MDA-MB-468 xenografts. Tumor fragments (˜1.5 mm³) were excised from subcutaneous donor tumors, rinsed in ice-cold phosphate-buffered saline to remove residual blood, and loaded into a 12-gauge trocar. The right flank of each recipient mouse was sterilized with 75% (v/v) ethanol, and a single fragment was implanted subcutaneously through a 3-mm incision that was closed with a sterile wound clip. When tumors reached an average volume of ˜100 mm³ (day 0), mice were randomly assigned (n=6 per group) to receive intraperitoneal injections of vehicle (10% DMSO in corn oil, 100 μL), ID230301A-1 (5 mg kg⁻¹), or paclitaxel (5 mg kg⁻¹) once daily for 24 consecutive days. Body weight and tumor dimensions were measured every other day using digital calipers. On day 25, mice were euthanized by CO₂ asphyxiation followed by cervical dislocation; tumors were excised and weighed immediately.

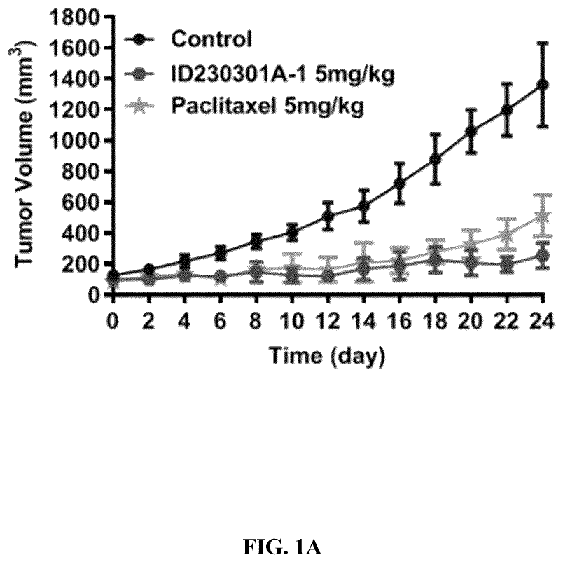

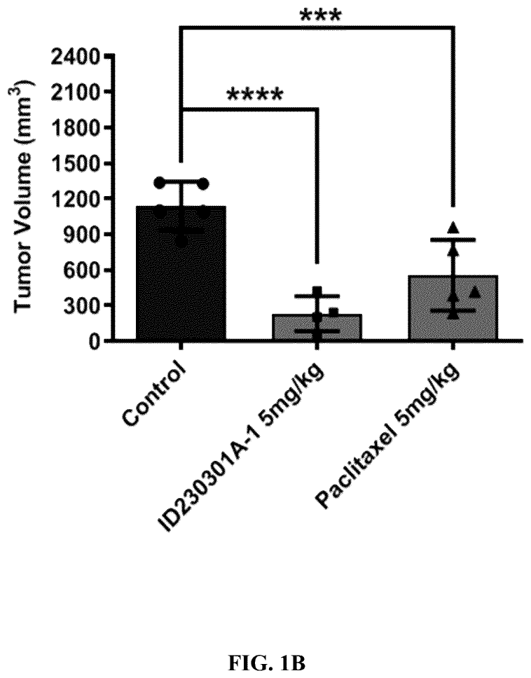

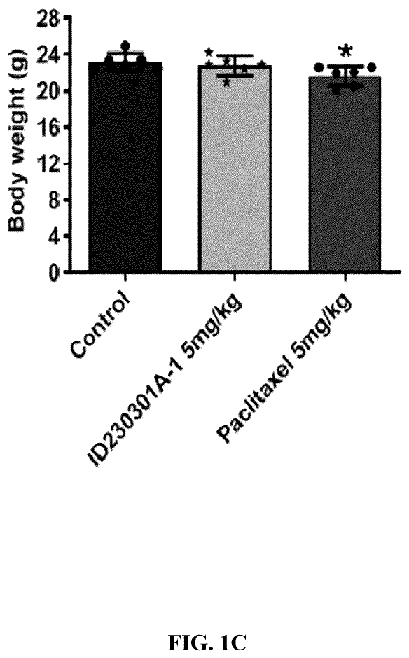

As shown in FIG. 1A, both ID230301A-1 (5 mg kg⁻¹, i.p.) and paclitaxel (5 mg kg⁻¹, i.p.) elicited profound and durable suppression of MDA-MB-468 xenograft growth relative to vehicle-treated controls. Quantitative end-point analysis revealed that ID230301A-1 conferred significantly greater antitumor efficacy than paclitaxel (FIG. 1B). At termination, paclitaxel caused significant weight loss versus vehicle, whereas ID230301A-1 preserved normative body-mass (FIG. 1C). Throughout the study, ID230301A-1 was well tolerated and did not elicit any observable treatment-related adverse events. Collectively, these data demonstrate that ID230301A-1 achieves superior antitumor potency coupled with an improved tolerability profile relative to paclitaxel in this xenograft model.

### Example 4

### Evaluation of STAT3 Signaling Modulation by ID230301A-1 Using Western Blot

MGC803 cells in the logarithmic growth phase were seeded into a six-well plates at a density of 6×10⁵ cells per well. After cell attachment, ID230301A-1 was added at final concentrations of 0, 30, 100, 300 and 1000 nM, respectively. Following a 24-hour incubation, cells were lysed using RIPA buffer, and protein extracts were subjected to Western blot analysis. The expression levels of STAT3, p-STAT3 (Tyr705), p-STAT3 (Ser727) and GAPDH were determined using the corresponding antibodies.

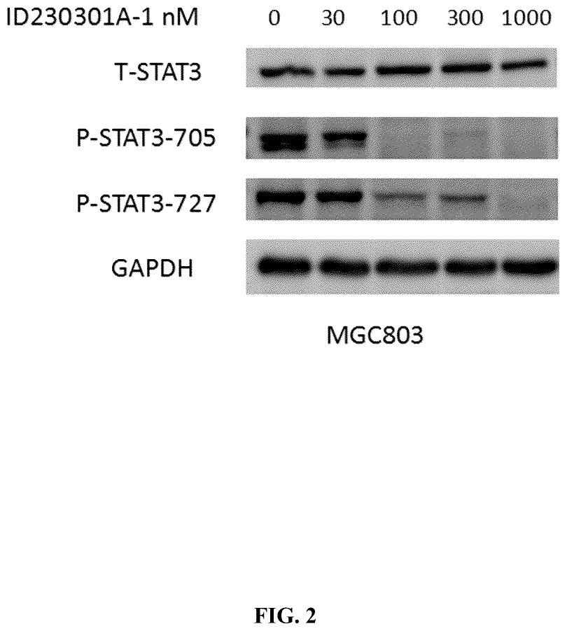

As shown in FIG. 2, the expression levels of p-STAT3 (Tyr705) and p-STAT3 (Ser727) in MGC803 cells were significantly inhibited when the concentration of ID230301A-1 was increased to 1000 nM compared to the solvent-treated controls. When the concentration of ID230301A-1 was increased to 1000 nM, the expression of both p-STAT3 (Tyr705) and p-STAT3 (Ser727) was completely inhibited.

Overall results show that ID230301A-1, ID230302A-1, ID230303A-1, ID230304A-1, ID230305A-1, ID230306A-1, ID230307A-1, ID230308A-1, ID230309A-1, ID230310A-1, ID230311A-1, ID230312A-1, ID230313A-1, ID230314A-1 and ID230315A-1 can significantly inhibit lung cancer, breast cancer, liver cancer, pancreatic cancer, stomach cancer, thyroid cancer, glioma, head cancer, esophageal cancer, gall cancer, colon cancer, testicular cancer, thymoma, renal cancer, prostate cancer, bladder cancer, uterine cancer, ovarian cancer, retinoblastoma, mesothelioma, osteosarcoma, lymphoma, multiple myeloma, leukemia, chronic bone marrow proliferation abnormality syndrome and proliferation of melanoma cells, and that ID230301A-1 at equivalent doses has better efficacy and safety in mice compared to the chemotherapeutic alpoprotein-binding cyanide.

In accordance with the general route of drug development (first conventional in vitro anti-tumor screening and then targeted research), the compounds of the disclosure can be applied to cancer therapeutic drugs associated with cell proliferation abnormalities, which can be prepared as anti-tumor drugs by salinization acceptable to the human body or by mixing with medicinal carriers.

Finally, it should be noted that the above embodiments are intended only to illustrate and not to limit the technical scheme of the disclosure, and any equivalent replacement of the disclosure and any modification or partial replacement that does not depart from the spirit and scope of the disclosure shall be covered within the scope of the protection of claims of the disclosure.

What is claimed is:

1. A compound having a general formula (I): 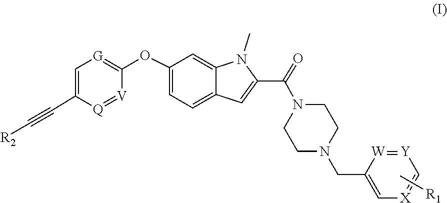
wherein:
R₁ is selected from the group consisting of H, F, Cl, Br, I, —CN, —CH₃, —CF₃, —OCH₃, —OCF₃, 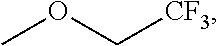
and —SO₂NH₂;
R₂ is selected from the group consisting of 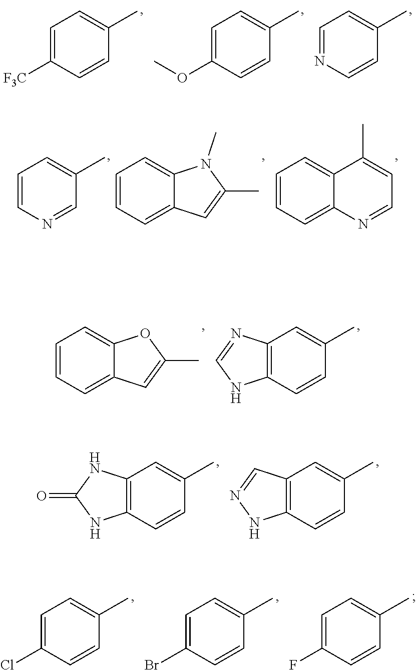
and
W, X, Y, G, Q, and V are each independently selected from the group consisting of C and N.

2. The compound of claim 1, being selected from the group consisting of the following formulas:

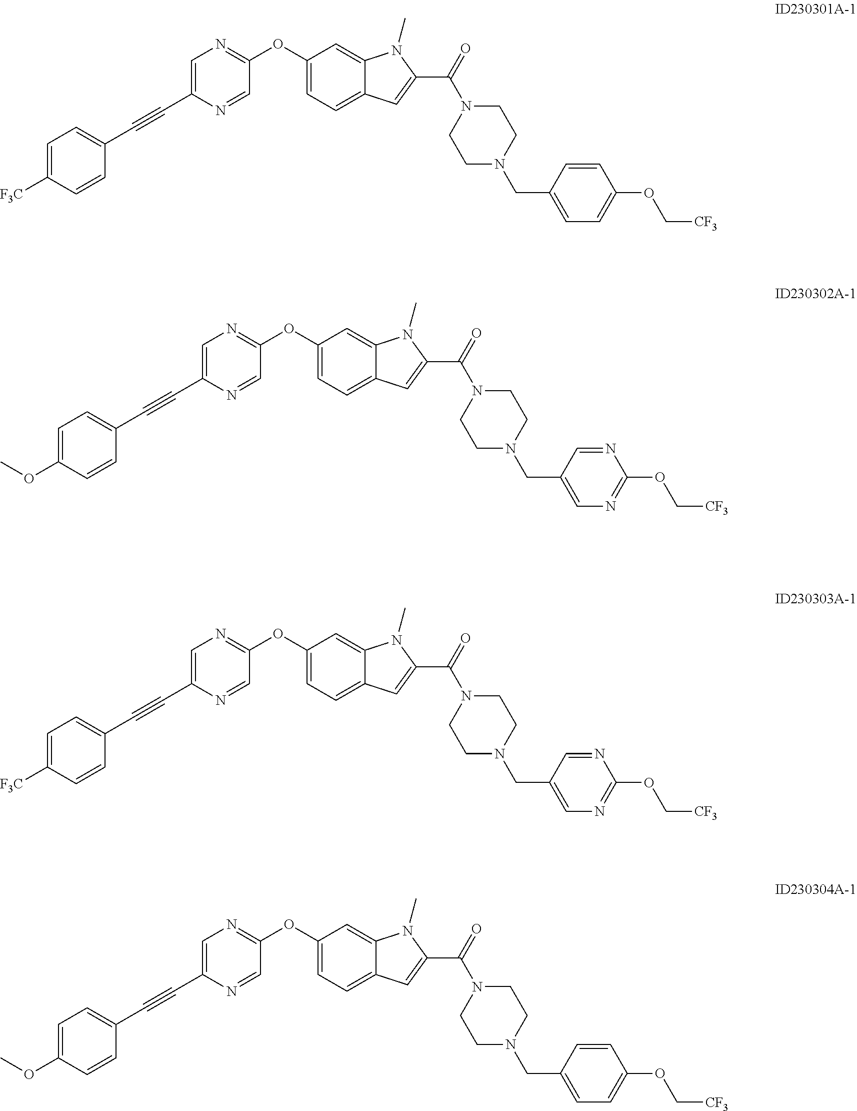 [SMILES: Cn1c(C(=O)N2CCN(Cc3ccc(OCC(F)(F)F)cc3)CC2)cc2ccc(Oc3cnc(C#Cc4ccc(C(F)(F)F)cc4)cn3)cc21 ; COc1ccc(C#Cc2cnc(Oc3ccc4cc(C(=O)N5CCN(Cc6cnc(OCC(F)(F)F)nc6)CC5)n(C)c4c3)cn2)cc1 ; Cn1c(C(=O)N2CCN(Cc3cnc(OCC(F)(F)F)nc3)CC2)cc2ccc(Oc3cnc(C#Cc4ccc(C(F)(F)F)cc4)cn3)cc21 ; COc1ccc(C#Cc2cnc(Oc3ccc4cc(C(=O)N5CCN(Cc6ccc(OCC(F)(F)F)cc6)CC5)n(C)c4c3)cn2)cc1]

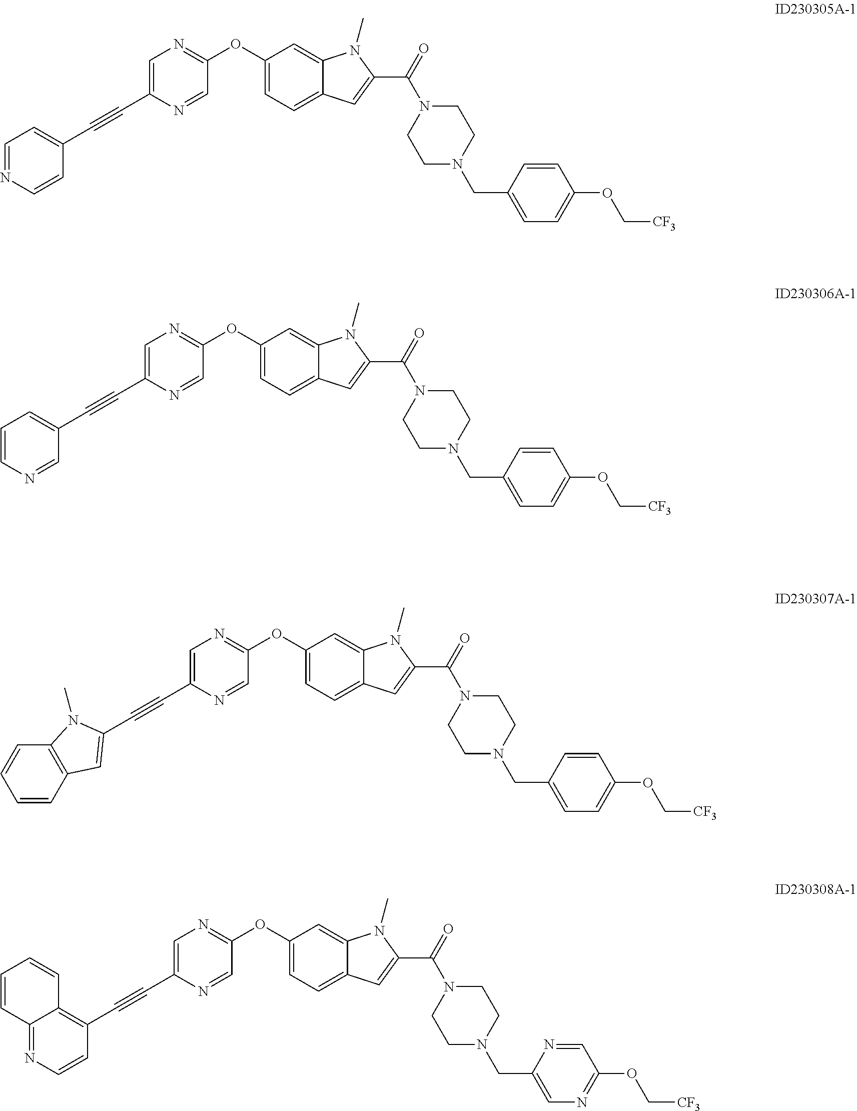 [SMILES: Cn1c(C(=O)N2CCN(Cc3ccc(OCC(F)(F)F)cc3)CC2)cc2ccc(Oc3cnc(C#Cc4ccncc4)cn3)cc21 ; Cn1c(C(=O)N2CCN(Cc3ccc(OCC(F)(F)F)cc3)CC2)cc2ccc(Oc3cnc(C#Cc4cccnc4)cn3)cc21 ; Cn1c(C#Cc2cnc(Oc3ccc4cc(C(=O)N5CCN(Cc6ccc(OCC(F)(F)F)cc6)CC5)n(C)c4c3)cn2)cc2ccccc21 ; Cn1c(C(=O)N2CCN(Cc3cnc(OCC(F)(F)F)cn3)CC2)cc2ccc(Oc3cnc(C#Cc4ccnc5ccccc45)cn3)cc21]

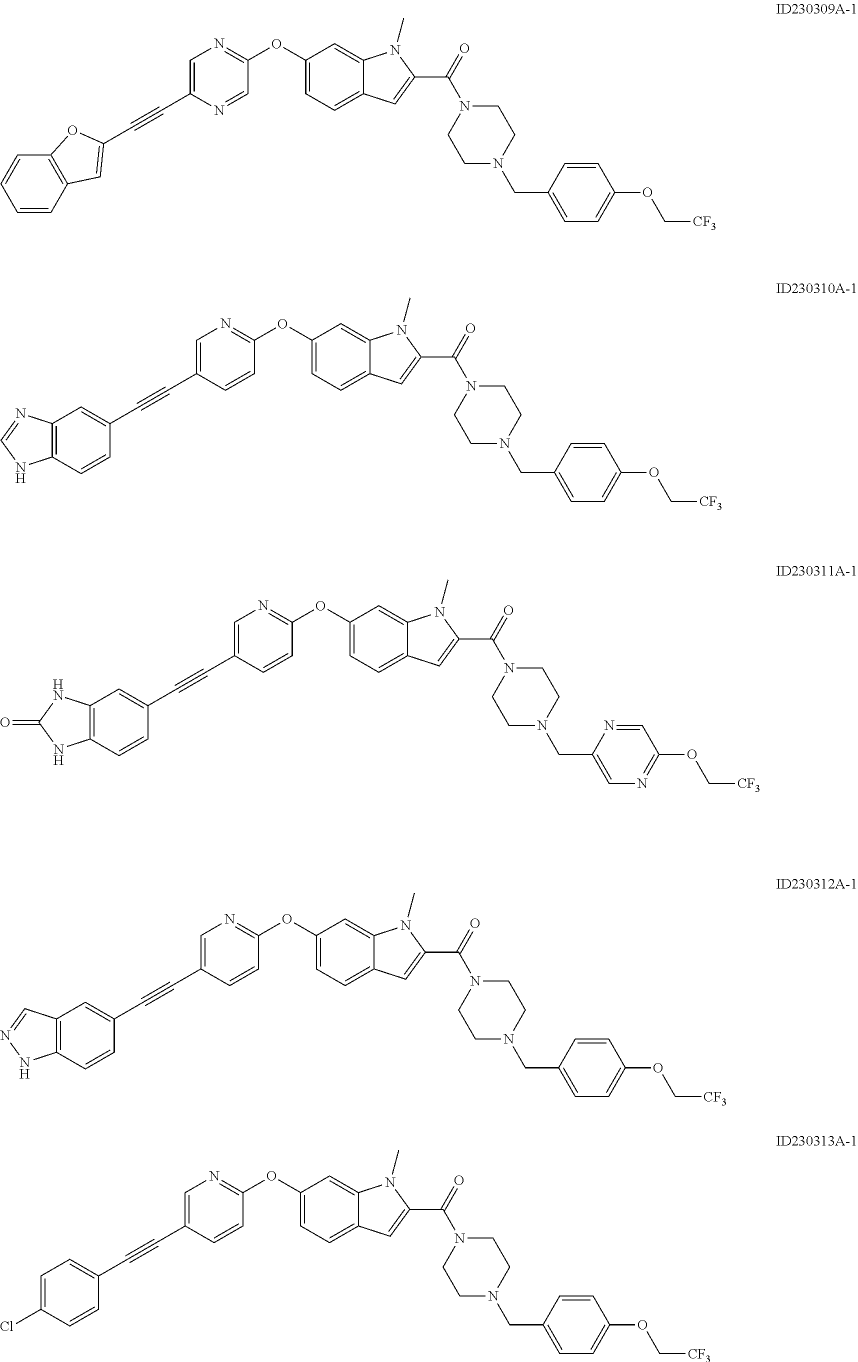 [SMILES: Cn1c(C(=O)N2CCN(Cc3ccc(OCC(F)(F)F)cc3)CC2)cc2ccc(Oc3cnc(C#Cc4cc5ccccc5o4)cn3)cc21 ; Cn1c(C(=O)N2CCN(Cc3ccc(OCC(F)(F)F)cc3)CC2)cc2ccc(Oc3ccc(C#Cc4ccc5[nH]cnc5c4)cn3)cc21 ; Cn1c(C(=O)N2CCN(Cc3cnc(OCC(F)(F)F)cn3)CC2)cc2ccc(Oc3ccc(C#Cc4ccc5[nH]c(=O)[nH]c5c4)cn3)cc21 ; Cn1c(C(=O)N2CCN(Cc3ccc(OCC(F)(F)F)cc3)CC2)cc2ccc(Oc3ccc(C#Cc4ccc5[nH]ncc5c4)cn3)cc21 ; Cn1c(C(=O)N2CCN(Cc3ccc(OCC(F)(F)F)cc3)CC2)cc2ccc(Oc3ccc(C#Cc4ccc(Cl)cc4)cn3)cc21]

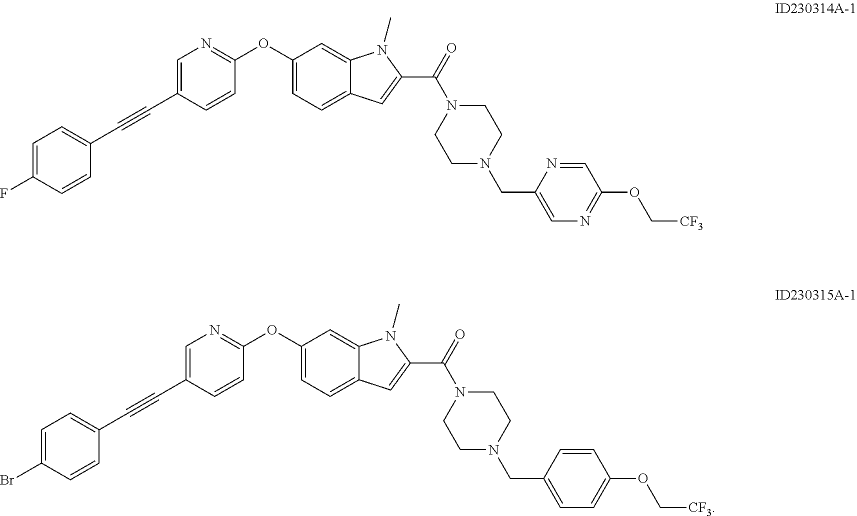 [SMILES: Cn1c(C(=O)N2CCN(Cc3cnc(OCC(F)(F)F)cn3)CC2)cc2ccc(Oc3ccc(C#Cc4ccc(F)cc4)cn3)cc21 ; Cn1c(C(=O)N2CCN(Cc3ccc(OCC(F)(F)F)cc3)CC2)cc2ccc(Oc3ccc(C#Cc4ccc(Br)cc4)cn3)cc21]

3. A pharmaceutically acceptable salt of the compound of claim 1, the salt being prepared by contacting the compound with at least one acid selected from the group consisting of acetic acid, dihydrofolic acid, benzoic acid, citric acid, sorbic acid, propionic acid, oxalic acid, fumaric acid, maleic acid, hydrochloric acid, malic acid, phosphoric acid, sulfurous acid, sulfuric acid, vanillic acid, tartaric acid, ascorbic acid, boric acid, lactic acid, and ethylenediaminetetraacetic acid.

4. A method of preparing the compound of claim 1 with a synthetic route as follows: 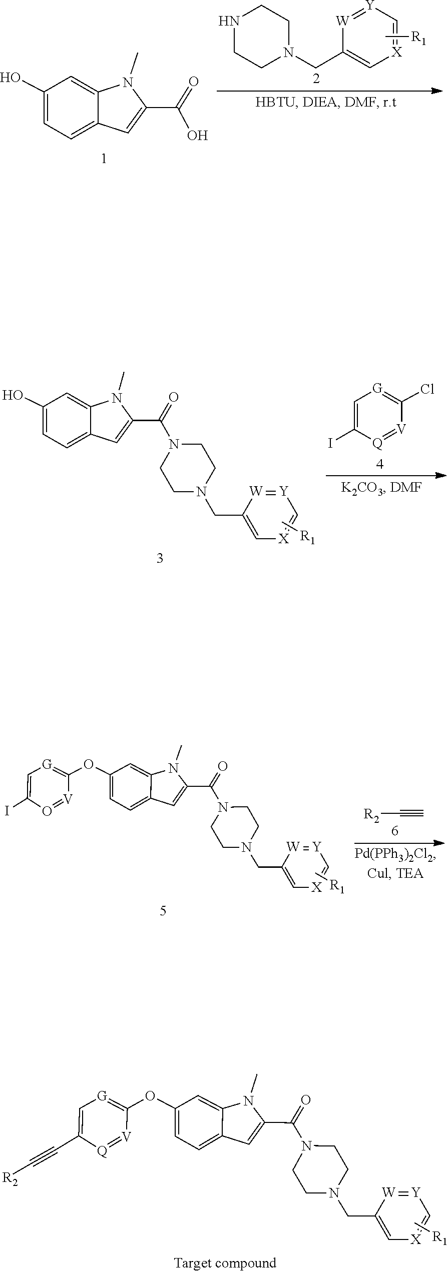
the method comprising:
(a) dissolving a first mixture of Compound 1, Compound 2, HBTU and DIEA in DMF, stirring the first mixture at a temperature of 20° C. to 30° C., diluting the first mixture with EA and washing with brine, collecting a first organic layer and concentrating under reduced pressure to yield a first crude product, and triturating the first crude product with EA to yield Compound 3;
(b) dissolving a second mixture of Compound 3, Compound 4 and K₂CO₃ in DMF, stirring the second mixture at a temperature of 75° C. to 85° C., diluting the second mixture with EA and washing with brine, collecting a second organic layer and concentrating under reduced pressure to yield a second crude product, and triturating the second crude product with EA to yield Compound 5; and
(c) stirring a third mixture of Compound 5, Compound 6, Pd(PPh₃)₂Cl₂, CuI, and TEA under a nitrogen atmosphere at a temperature of 75° C. to 85° C., diluting the third mixture with EA and washing with brine, collecting a third organic layer and concentrating under reduced pressure to yield a third crude product, triturating the third crude product with EA to yield a target compound.

5. The method of claim 4, wherein:
a molar ratio of Compound 1 to Compound 2 to HBTU to DIEA is 1:1:1.2:3 in (a);
a molar ratio of Compound 3 to Compound 4 to K₂CO₃ is 1:1:1.2 in (b); and
a molar ratio of Compound 5 to Compound 6 to Pd(PPh₃)₂Cl₂ to CuI to TEA is 1:5:0.1:0.2:5 in (c).

6. A method for treating a disease associated with STAT3 signaling, the method comprising administering a subject in need thereof the compound of claim 1 or a pharmaceutically acceptable salt thereof.

7. The method of claim 6, wherein the disease is a cancer.

8. The method of claim 6, wherein the cancer is selected from the group consisting of lung cancer, breast cancer, liver cancer, pancreatic cancer, gastric cancer, thyroid cancer, glioma, head and neck cancer, esophageal cancer, cholangiocarcinoma, colorectal cancer, testicular cancer, thymoma, renal cancer, prostate cancer, bladder cancer, uterine cancer, ovarian cancer, retinoblastoma, mesothelioma, osteosarcoma, lymphoma, multiple myeloma, leukemia, chronic myelodysplastic syndrome, and melanoma.
